(19) United States
(12) Patent Application Publication
(10) Pub. No.: US 2024/0173323 A1
(43) Pub. Date: May 30, 2024

(54) COMBINATION THERAPY FOR CANCER TREATMENT

(71) Applicant: Takeda Pharmaceutical Company Limited, Osaka (JP)

(72) Inventors: Akihiro OHASHI, Kanagawa (JP); Kenichi IWAI, Kanagawa (JP); Tadahiro NAMBU, Kanagawa (JP); Jie YU, Cambridge, MA (US); Kurt ENG, Cambridge, MA (US); Michael Joseph KURANDA, Cambridge, MA (US)

(21) Appl. No.: 18/264,489

(22) PCT Filed: Feb. 4, 2022
(86) PCT No.: PCT/IB2022/050999

§ 371 Date: Aug. 7, 2023

Related U.S. Application Data
(60) Provisional application No. 63/146,919, filed on Feb. 8, 2021.

(51) Int. Cl. A61K 31/519; A61K 31/454; A61K 31/502; A61P 35/00

(52) U.S. Cl. CPC A61K 31/519; A61K 31/454; A61K 31/502; A61P 35/00

(57) ABSTRACT

The present disclosure relates to the treatment of cancer using a combination therapy comprising Compound 1 and/or tautomers thereof or a pharmaceutically acceptable salt or hydrate thereof, and one or more PARP inhibitors. Compound I.

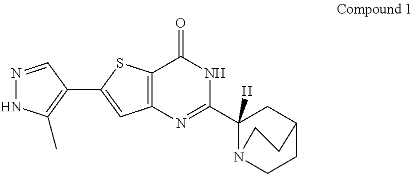 [SMILES: Cc1[nH]ncc1-c1cc2nc([C@@H]3CC4CCN3CC4)[nH]c(=O)c2s1]

### FIELD

The present disclosure relates to the treatment of cancer using a combination therapy comprising (i) Compound 1

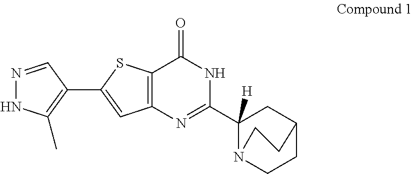 [SMILES: Cc1[nH]ncc1-c1cc2nc([C@@H]3CC4CCN3CC4)[nH]c(=O)c2s1]

and/or tautomers thereof or a pharmaceutically acceptable salt or hydrate thereof, and (ii) one or more PARP inhibitors.

### BACKGROUND

CDC7 is a serine/threonine kinase, which contributes to initiation of DNA replication by phosphorylating MCM2. Kinase activity of CDC7 is controlled by its binding protein Dbf4 in a cell-cycle dependent manner. Recent studies revealed that CDC7 is also involved in DNA damage response (DDR) as well as DNA replication, suggesting that CDC7 plays important roles in both cell proliferation during the S phase and genomic stability in DDR. Furthermore, elevated CDC7 expression has been reported in various cancers and correlates with poor prognosis, such as in diffuse large B cell lymphoma, oral squamous carcinoma, breast tumor, colon tumor, ovarian tumor and lung tumor.

Given that CDC7 is responsible for two key functions of DNA replication and DDR, CDC7 appears to be a critical gene for proliferation and survival of cancer cells and inhibition of CDC7 is expected to induce anti-proliferation and apoptosis in broad range of cancers, not limited to specific organ types of cancers. There is a need for new cancer therapies, such as combination therapies comprising CDC7 inhibitors.

### SUMMARY

In one aspect, the present disclosure provides a method for treating cancer in a patient in need thereof, comprising administering to the patient a therapeutically effective amount of Compound 1

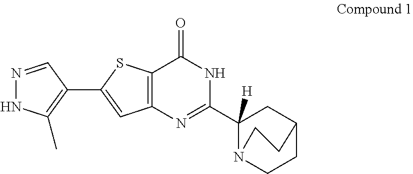 [SMILES: Cc1[nH]ncc1-c1cc2nc([C@@H]3CC4CCN3CC4)[nH]c(=O)c2s1]

and/or tautomers thereof or a pharmaceutically acceptable salt or hydrate thereof, and one or more PARP inhibitors.

In some embodiments, the PARP inhibitor is niraparib.

In some embodiments, the PARP inhibitor is olaparib.

In some embodiments, the cancer is ovarian cancer.

In some embodiments, the cancer is breast cancer.

Another aspect of the present disclosure provides a pharmaceutical composition comprising Compound 1

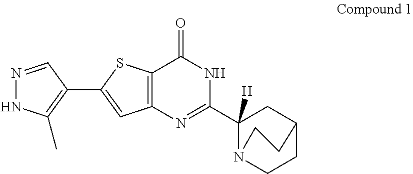 [SMILES: Cc1[nH]ncc1-c1cc2nc([C@@H]3CC4CCN3CC4)[nH]c(=O)c2s1]

and/or tautomers thereof or a pharmaceutically acceptable salt or hydrate thereof; and one or more PARP inhibitors.

In some embodiments, the pharmaceutical composition further comprises a pharmaceutically acceptable excipient.

Another aspect of the present disclosure provides Compound 1

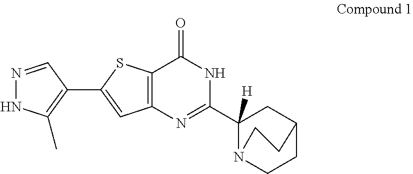 [SMILES: Cc1[nH]ncc1-c1cc2nc([C@@H]3CC4CCN3CC4)[nH]c(=O)c2s1]

and/or tautomers thereof or a pharmaceutically acceptable salt or hydrate thereof for use in combination with one or more PARP inhibitors for the treatment of cancer.

Another aspect of the present disclosure provides use of Compound 1

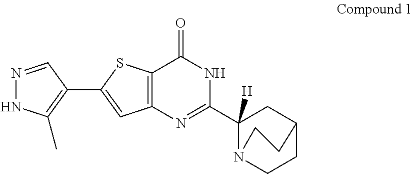 [SMILES: Cc1[nH]ncc1-c1cc2nc([C@@H]3CC4CCN3CC4)[nH]c(=O)c2s1]

and/or tautomers thereof or a pharmaceutically acceptable salt or hydrate thereof in combination with one or more PARP inhibitors for the treatment of cancer.

### BRIEF DESCRIPTION OF FIGURES

FIG. 1 shows that Compound 1 combined with niraparib exhibits strong antitumor activity compared to either single treatment alone against PHTXS-13O human primary ovarian cancer xenografts.

FIG. 2 shows that Compound 1 combined with olaparib exhibits strong antitumor activity compared to either single treatment alone against PHTXS-130O human primary ovarian cancer xenografts.

FIG. 3 shows the combinational antitumor activity of Compound 1 and niraparib in Balb/c nude mice bearing PHTXS-130 human primary ovarian cancer xenografts.

### DETAILED DESCRIPTION

Unless defined otherwise, all technical and scientific terms used herein have the same meaning as commonly understood by one of ordinary skill in the art to which this disclosure belongs. Accordingly, the following terms are intended to have the following meanings:

As used in the specification and claims, the singular form “a”, “an” and “the” includes plural references unless the context clearly dictates otherwise.

As used herein, “administration” of a disclosed compound encompasses the delivery to a subject of a compound as described herein, or a prodrug or other pharmaceutically acceptable derivative thereof, using any suitable formulation or route of administration, e.g., as described herein.

As used herein, “effective amount” or “therapeutically effective amount” refers to the amount of a compound or pharmaceutical composition described herein that is sufficient to effect the intended application including, but not limited to, disease treatment, as illustrated below. In some embodiments, the amount is that effective for detectable killing or inhibition of the growth or spread of cancer cells; the size or number of tumors; or other measure of the level, stage, progression or severity of the cancer. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of cell migration. The specific dose will vary depending on, for example, the particular compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

As used herein, “treatment” and “treating”, are used interchangeably herein, and refer to an approach for obtaining beneficial or desired results including, but not limited to, therapeutic benefit. By therapeutic benefit is meant eradication or amelioration of the underlying disorder being treated. Also, a therapeutic benefit is achieved with the eradication or amelioration of one or more the physiological symptoms associated with the underlying disorder such that an improvement is observed in the patient, notwithstanding that the patient can still be afflicted with the underlying disorder.

As used herein, “subject” or “patient” to which administration is contemplated includes, but is not limited to, humans (i.e., a male or female of any age group) or other primates.

The term “comprises or comprising” refers to “includes, but is not limited to.”

The present disclosure provides methods for treating cancer in a patient in need of treatment. The methods comprise administering to a patient in need thereof a therapeutically effective amount of (i) Compound 1

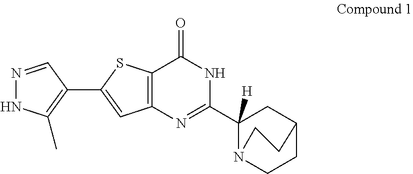 [SMILES: Cc1[nH]ncc1-c1cc2nc([C@@H]3CC4CCN3CC4)[nH]c(=O)c2s1]

and/or tautomers thereof or a pharmaceutically acceptable salt or hydrate thereof, and (ii) one or more poly(ADP-ribose)polymerase (PARP) inhibitors.

The present disclosure further provides a pharmaceutical composition comprising Compound 1 and/or tautomers thereof or a pharmaceutically acceptable salt or hydrate thereof and PARP inhibitors.

The present disclosure further provides a pharmaceutical combination comprising a composition comprising Compound 1 and/or tautomers thereof or a pharmaceutically acceptable salt or hydrate thereof and a composition comprising PARP inhibitor.

The present disclosure further provides a kit comprising an article for sale containing a combination comprising Compound 1 and/or tautomers thereof or a pharmaceutically acceptable salt or hydrate thereof and PARP inhibitor, each separately packaged with instructions for use to treat cancer.

The combination therapies of the present disclosure include Compound 1 and/or tautomers thereof or a pharmaceutically acceptable salt or hydrate thereof. Compound 1 has the following structure:

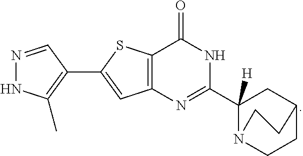 [SMILES: Cc1[nH]ncc1-c1cc2nc([C@@H]3CC4CCN3CC4)[nH]c(=O)c2s1]

The chemical name for Compound 1 is 2-[(2S)-1-azabicyclo[2.2.2]oct-2-yl]-6-(3- methyl-1H-pyrazol-4-yl)thieno[3,2-d]pyrimidin-4(3H)-one. Compound 1 is a Cdc7 kinase inhibitor.

CDC7 inhibitors other than Compound 1 are also expected to show good antitumor efficacy in combination with a PARP inhibitor. Thus, in alternative embodiments, the present disclosure further provides a combination therapy comprising (i) a CDC7 kinase inhibitor other than Compound 1 and (ii) a PARP inhibitor. In some embodiments, the CDC7 kinase inhibitor may be selected from LY3143921, KC-459, MSK-777 or RXDX-103. Accordingly, the present disclosure also provides a method for treating cancer in a patient in need thereof, comprising administering to the patient a therapeutically effective amount of a CDC7 kinase inhibitor and one or more PARP inhibitors.

Tautomers of Compound 1 or a pharmaceutically acceptable salt or hydrate of Compound 1 are/is also encompassed by the present disclosure. When Compound 1 has a tautomer, each isomer is also encompassed in the present disclosure.

As used herein the phrases “Compound 1 and/or tautomers thereof” and the like are all understood to mean Compound 1 and all of its tautomeric forms. As a non-limiting example, tautomerization may occur in the pyrazole and pyrimidine groups of Compound 1. Specific examples of tautomerization that may occur in Compound 1 include:

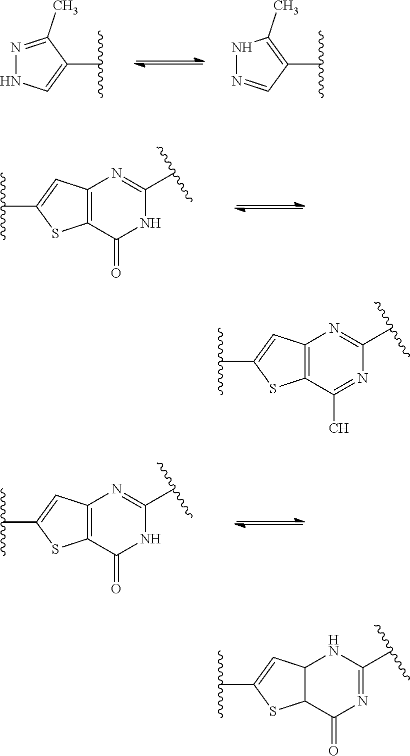

Compound 1 and/or tautomers thereof can be used in the form of a pharmaceutically acceptable salt. Examples of the pharmaceutically acceptable salt include salts with inorganic bases, salts with organic bases, salts with inorganic acids, salts with organic acids, and salts with basic or acidic amino acids.

Compound 1 and/or tautomers thereof may be a hydrate (e.g., hemihydrate), a non- hydrate, a solvate or a non-solvate, all of which are encompassed in the present disclosure. In some embodiments, Compound 1 and/or tautomers thereof is a hemihydrate.

Compound 1 and/or tautomers thereof or a pharmaceutically acceptable salt or hydrate thereof or a crystal form thereof can be obtained according to the production methods described in PCT Publication No. WO 2011/102399, U.S. Pat. No. 8,722,660, U.S. Pat. No. 8,921,354, U.S. Pat. No. 8,933,069, and U.S. Patent Publication No. US 2015/158882, which are incorporated herein by reference in their entirety and for all purposes, or a method analogous thereto.

Compound 1 and/or tautomers thereof or a pharmaceutically acceptable salt or hydrate thereof may be in the form of a crystal (e.g., crystalline form A, crystalline form I, etc.), and the crystal form of the crystal may be single or plural, both of which are encompassed in Compound 1. The crystal may be of a form, and can be produced by a method, described in PCT publication no. WO 2017/172565, published Oct. 5, 2017, which is incorporated herein by reference in its entirety for all purposes. In some embodiments, the Compound 1 and/or tautomers thereof or a pharmaceutically acceptable salt or hydrate thereof may be in the form of Crystalline Form I as described in WO 2017/172565. In some embodiments, the Compound 1 and/or tautomers thereof or a pharmaceutically acceptable salt or hydrate thereof is a crystalline form of Compound 1 hemihydrate (i.e., 2-[(2S)-1- azabicyclo[2.2.2]oct-2-yl]-6-(3-methyl-lH-pyrazol-4- ypthieno[3,2-d]pyrimidin-4(3H)-one hemihydrate). For example, the Compound 1 and/or tautomers thereof or a pharmaceutically acceptable salt or hydrate thereof may be Crystalline Form I of Compound 1 hemihydrate.

In some embodiments, the combination therapy includes PARP inhibitor.

In some embodiments, the PARP inhibitor is selected from the group consisting of niraparib, olaparib, veliparib, rucaparib, pamiparib, iniparib and terazoparib.

In certain embodiments, the PARP inhibitor is niraparib.

In certain embodiments, the PARP inhibitor is olaparib.

The Examples section below describes in vivo studies of combination therapies using PARP inhibitor. Numerous PARP inhibitors were tested on cancer cell lines. The results demonstrate the efficacy of the combination therapies of the present disclosure.

In some embodiments, Compound 1 and/or tautomers thereof or a pharmaceutically acceptable salt or hydrate thereof and PARP inhibitor may be formulated as a pharmaceutical composition with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, PA, 1995.

Pharmaceutical compositions used in embodiments of the present disclosure may also include diluents, fillers, salts, buffers, detergents (e. g., a nonionic detergent, such as Tween- 80), stabilizers (e. g., sugars or protein-free amino acids), preservatives, tissue fixatives, solubilizers, and/or other materials suitable for inclusion in a pharmaceutical composition.

The compounds used in embodiments of the present disclosure may be administered via any suitable route, such as an oral, nasal, inhalable, topical (including buccal, transdermal and sublingual), rectal, vaginal and/or parenteral route.

In certain embodiments, one or more the compounds used in the present disclosure are administered orally, for example, with an inert diluent or an assimilable edible carrier. The active ingredient may be enclosed in a hard or soft shell gelatin capsule, or compressed into tablets. Pharmaceutical compositions which are suitable for oral administration include ingestible tablets, buccal tablets, troches, capsules, elixirs, suspensions, syrups, wafers, and the like containing such carriers as are known in the art to be appropriate.

In certain embodiments, one or more the compounds used in the present disclosure are administered parenterally. The phrases “parenteral administration” and “administered parenterally” as used herein mean modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

The methods of this disclosure provide efficacious treatments for patients with cancer. In some embodiments, the cancer treated with the combination therapy of the present disclosure is a cancer mediated by Cdc7 (for example, colorectal cancer (e.g., metastatic colorectal cancer), lung cancer (e.g., non-small cell lung cancer (e.g., squamous non-small cell lung cancer (including locally advanced squamous non-small cell lung cancer and metastatic squamous non-small cell lung cancer)), mesothelioma, pancreatic cancer (e.g., metastatic pancreatic cancer), pharyngeal cancer, laryngeal cancer, esophageal cancer (e.g., squamous esophageal cancer), gastric cancer duodenal cancer, small intestinal cancer, breast cancer, ovarian cancer, testis tumor, prostate cancer, liver cancer, thyroid cancer, kidney cancer, uterine cancer, brain tumor, retinoblastoma, skin cancer, bone tumor, urinary bladder cancer, hematologic cancer (e.g., multiple myeloma, leukemia, malignant lymphoma, Hodgkin's disease, chronic bone marrow proliferative disease).

In some embodiments, the cancer treated with the combination therapy of this disclosure is selected from the group consisting of lung cancer (e.g., non-small cell lung cancer (e.g., squamous non-small cell lung cancer including locally advanced squamous non- small cell lung cancer and metastatic squamous non-small cell lung cancer)), colorectal cancer (e.g., metastatic colorectal cancer), ovarian cancer, breast cancer and pancreatic cancer (e.g., metastatic pancreatic cancer).

In some embodiments, the cancer treated with the combination therapy of this disclosure is ovarian cancer. In some embodiments, the cancer treated with the combination therapy of this disclosure is breat cancer

In some embodiments, the dose strength of Compound 1 and/or tautomers thereof or a pharmaceutically acceptable salt or hydrate thereof ranges from 5 to 200 mg. For example, in some embodiments, a medicament comprises a dose strength of 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 mg of Compound 1 and/or tautomers thereof or a pharmaceutically acceptable salt or hydrate thereof. In some embodiments, the daily dose of Compound 1 and/or tautomers thereof or a pharmaceutically acceptable salt or hydrate thereof administered to an adult (body weight about 60 kg) ranges from 10 to 200 mg. In other embodiments, the daily dose to an adult of Compound 1 and/or tautomers thereof or a pharmaceutically acceptable salt or hydrate thereof is about 1 to 1000 mg, about 3 to 300 mg, or about 10 to 200 mg, which can be given in a single administration or administered in 2 or 3 portions a day. In some embodiments, the Compound 1 and/or tautomers thereof or a pharmaceutically acceptable salt or hydrate thereof is administered orally.

In some embodiments, the combination therapy comprises niraparib, wherein niraparib is administered at a dose from about 100 mg/day to about 300 mg/day. In some embodiments, niraparib is administered orally. In some embodiments, niraparib is administered daily.

In some embodiments, the combination therapy comprises olaparib, wherein olaparib is administered at a dose from about 400 mg/day to about 600 mg/day. In some embodiments, olaparib is administered orally. In some embodiments, olaparib is administered daily.

In certain embodiments, the Compound 1 and/or tautomers thereof or a pharmaceutically acceptable salt or hydrate thereof is administered orally. In some embodiments, the Compound 1 and/or tautomers thereof or a pharmaceutically acceptable salt or hydrate thereof is administered daily, once every two days, once every three days, once every four days, once every five days, once every six days, once a week, once every two weeks, or once every four weeks.

In certain embodiments, the Compound 1 and/or tautomers thereof or a pharmaceutically acceptable salt or hydrate thereof and the PARP inhibitor may be administered simultaneously or sequentially in any order. In certain embodiments, they may be administered separately or together in one or more pharmaceutical compositions.

In some embodiments, Compound 1 and/or tautomers thereof or a pharmaceutically acceptable salt or hydrate thereof can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of PARP inhibitor to patients with cancer.

In some embodiments, the combination therapy comprises a 28 day cycle wherein Compound 1 and/or tautomers thereof or a pharmaceutically acceptable salt or hydrate thereof is administered once daily on days 1-28 and niraparib is administered once daily on days 1-28.

In some embodiments, the combination therapy comprises a 28 day cycle wherein Compound 1 and/or tautomers thereof or a pharmaceutically acceptable salt or hydrate thereof is administered once daily on days 1-28 and olaparib is administered twice daily on days 1-28.

### EXAMPLES

### Example 1 In Vivo Antitumor Activity of Compound 1 in combination with PARP inhibitor in Patient Derived Xenograft (PDX) model

To investigate in vivo antitumor activity of Compound 1 in combination with PARP inhibitor, tests using Patient Derived Xenograft (PDX) model were conducted. Patient Derived Tumors were inoculated by the following Method.

Method: Patient Derived Tumors were maintained in nude mice by subcutaneous inoculation of tumor pieces (approx. 2×2×2 mm) into nude mice. Mice with tumor size of approximately 130 mm³ (e.g., 125-130 mm³) for xenograft studies were randomly assigned to dose groups on the day (Day 0) before start date of dosing.

Compound 1 (crystalline form I) was suspended in solution comprising 0.5 w/v % methylcellulose and administered orally to mice.

Concomitant drug was administered as shown in Table 2.

Tumor size was measured by caliper and tumor volume was estimated using the equation V=(LW²)/2, where L and W are tumor length and width, respectively and reported in cubic millimeters. The results of this study were shown in Table 2.

Statistical analyses of combination effect for tumor growth was conducted as follows;

All tumor values (tumor volumes or photon flux) had a value of 1 added to them before logio transformation. These values were compared across treatment groups to assess whether the differences in the trends over time were statistically significant. To compare pairs of treatment groups, the following mixed-effects linear regression model was fit to the data using the maximum likelihood method:

Yᵢⱼₖ−Yᵢ₀ₖ=Yᵢ₀ₖ treatᵢ+dayⱼ+dayⱼ²+(treat*day)ᵢⱼ+(treat*day²)ᵢⱼ+eᵢⱼₖ (1)

where Yijk is the log₁₀ tumor value at the j^{th} time point of the k^{th} animal in the i^{th} treatment,

Yᵢ₀ₖ is the day 0 (baseline) login tumor value in the k^{th} animal in the i^{th} treatment, dayⱼ was the median-centered time point and (along with day²ⱼ) was treated as a continuous variable, and eᵢⱼₖ is the residual error. A spatial power law covariance matrix was used to account for the repeated measurements on the same animal over time. Interaction terms as well as day²ⱼ terms were removed if they were not statistically significant.

A likelihood ratio test was used to assess whether a given pair of treatment groups exhibited differences which were statistically significant. The −2 log likelihood of the full model was compared to one without any treatment terms (reduced model) and the difference in the values

was tested using a Chi-squared test. The degrees of freedom of the test were calculated as the difference between the degrees of freedom of the full model and that of the reduced model. The predicted differences in the log tumor values (Yᵢⱼₖ-Yᵢₒₖ, which can be interpreted as log₁₀(fold change from day 0)) were taken from the above models to calculate mean AUC values for each treatment group. A dAUC value was then calculated as:

$$dAUC = \frac{mean ( {AUC}_{ctl} ) - mean ( {AUC}_{trt} )}{mean ( {AUC}_{ctl} )} * 100 \qquad ( 2 )$$

This assumed AUC_{ctl} was positive. In instances where AUC_{ctl} was negative, the above formula was multiplied by −1.

For synergy analyses, the observed differences in the log tumor values were used to calculate AUC values for each animal In instances when an animal in a treatment group was removed from the study, the last observed tumor value was carried forward through all subsequent time points. The AUC for the control, or vehicle, group was calculated using the predicted values from the pairwise models described above. We defined a measure of synergy as follows:

$${Frac}_{{A}_{k}} = \frac{{AUC}_{ctl} - {AUC}_{{A}_{k}}}{{AUC}_{ctl}} \qquad ( 3 )$$

$${Frac}_{{B}_{k}} = \frac{{AUC}_{ctl} - {AUC}_{{B}_{k}}}{{AUC}_{ctl}} \qquad ( 4 )$$

$${Frac}_{{AB}_{k}} = \frac{{AUC}_{ctl} - {AUC}_{{AB}_{k}}}{{AUC}_{ctl}} \qquad ( 5 )$$

$$synergy score = ( mean ( {Frac}_{A} ) + mean ( {Frac}_{B} ) - mean ( {Frac}_{AB} ) ) * 100 \qquad ( 6 )$$

where Aₖ and Bₖ are the h^{th} animal in the individual treatment groups and ABₖ is the h^{th} animal in combination treatment group. AUC_{dtl} is the model-predicted AUC for the control group and was treated as a constant with no variability. The standard error of the synergy score was calculated as the square root of the sum of squared standard errors across groups A, B, and AB. The degrees of freedom were estimated using the Welch-Satterthwaite equation. A hypothesis test was performed to determine if the synergy score differed from 0. P values were calculated by dividing the synergy score by its standard error and tested against a t- distribution (two-tailed) with the above-calculated degrees of freedom.

The effect was classified into four different categories. It was considered synergistic if the synergy score was less than 0 and additive if the synergy score wasn't statistically different from 0. If the synergy score was greater than zero, but the mean AUC for the combination was lower than the lowest mean AUC among the two single agent treatments, then the combination was sub-additive. If the synergy score was greater than zero, and the mean AUC for the combina- tion was greater than the mean AUC for at least one of the single agent treatments, then the combination was antagonistic.

Interval analysis, if requested, involved a specified treatment group and time interval com- pared with another treatment group and time interval. For a given group, time interval, and animal, the tumor growth rate per day was estimated by

Rate=100*(10^{ΔY/Δt}−1) (7)

where ΔY is the difference in the logio tumor volume over the interval of interest, and Δt is the length of the time interval. If one or both of the time points were missing, then the animal was ignored. The mean rates across the animals were then compared using a two-sided unpaired t-test with unequal variances.

Given the exploratory nature of this study, there were no adjustments pre-specified for the multiple comparisons and endpoints examined. All P values <0.05 were called statistically significant in this analysis.

TABLE 1
Inoculated cells in vivo study

| Name of Cell (Cancer type) | Provider |
|---|---|
| PHTXS-13O (Ovarian) | Hokkaido University (Sapporo, Japan) |

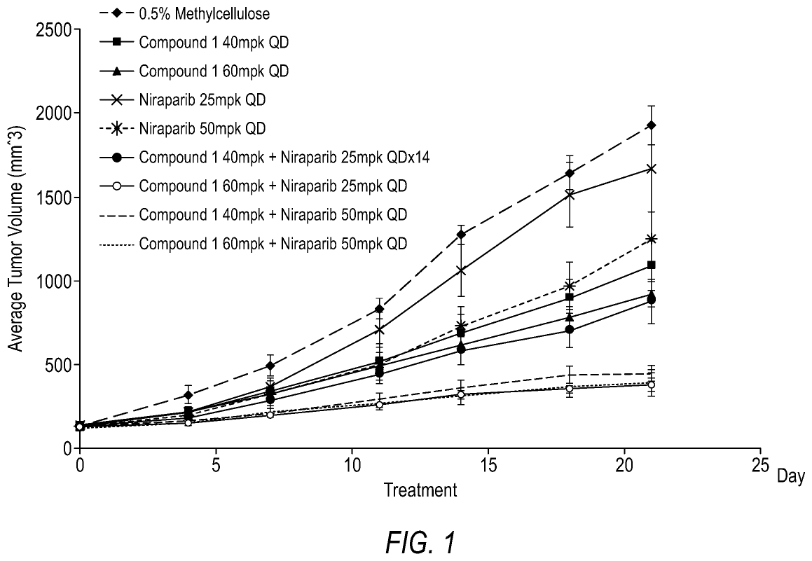

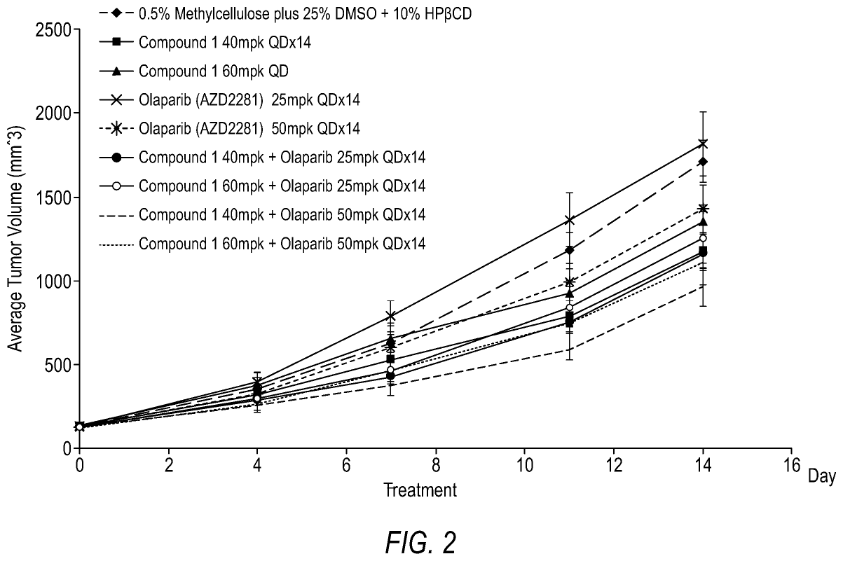

Change of average tumor volume after start of administration is described in FIGS. 1 and 2. Compound 1 showed good antitumor efficacy in combination with niraparib or olaparib.

TABLE 2
The syngeneic model study

| | Inoculated | | |
|---|---|---|---|
| | tumor cell | Compound 1 | |
| Concomitant | (starting | Administration | |
| drug | tumor size) | condition | Concomitant drug |
| Niraparib | PHTXS-13O | 40 mg/kg once | At 25 mg/kg daily, |
| | (130 mm³) | daily on Day 1-21 | orally on Days 1-21 |
| | | 60 mg/kg once | At 25 mg/kg daily, |
| | | daily on Day 1-21 | orally on Days 1-21 |
| | | 40 mg/kg once | At 50 mg/kg daily, |
| | | daily on Day 1-21 | orally on Days 1-21 |
| | | 60 mg/kg once | At 50 mg/kg daily, |
| | | daily on Day 1-21 | orally on Days 1-21 |
| Olaparib | PHTXS-13O | 40 mg/kg once | At 25 mg/kg daily, |
| | (125 mm³) | daily on Day 1-14 | orally on Days 1-14 |
| | | 60 mg/kg once | At 25 mg/kg daily, |
| | | daily on Day 1-14 | orally on Days 1-14 |
| | | 40 mg/kg once | At 50 mg/kg daily, |
| | | daily on Day 1-14 | orally on Days 1-14 |
| | | 60 mg/kg once | At 50 mg/kg daily, |
| | | daily on Day 1-14 | orally on Days 1-14 |

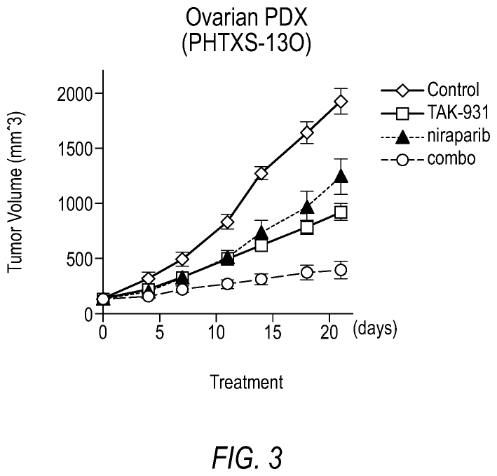

FIG. 3 illustrates another combinational antitumor activity of Compound 1 and PARP inhibitor, niraparib, in Balb/c nude mice bearing PHTXS-13O human primary ovarian cancer xenografts. The xenografted mice were administered with Compound 1 at 60 mg/kg and niraparib at 50 mg/kg, qd, for 21 days. Efficacy data plotted as the mean tumor volume (n=8) in vehicle control. Tumor size was continuously measured for the indicated periods after the termination of drug treatment.

1. A tablet comprising Compound 1 and/or tautomers thereof or a pharmaceutically acceptable salt or hydrate thereof 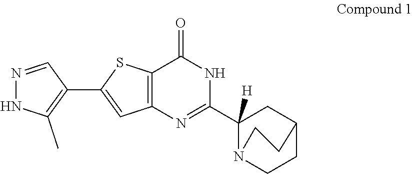 [SMILES: Cc1[nH]ncc1-c1cc2nc([C@@H]3CC4CCN3CC4)[nH]c(=O)c2s1]
and a compressible filler.

2. The tablet of claim 1, wherein the compressible filler is an intra-granular compressible filler present as an intra-granular component of the tablet; wherein the weight ratio of Compound 1 and/or tautomers thereof or a pharmaceutically acceptable salt or hydrate thereof to the intra-granular compressible filler is greater than 1:1.

3. The tablet of claim 2, wherein the weight ratio of Compound 1 and/or tautomers thereof or a pharmaceutically acceptable salt or hydrate thereof to the intra-granular compressible filler is from about 2:1 to about 10:1.

4. The tablet of claim 3, wherein the weight ratio of Compound 1 and/or tautomers thereof or a pharmaceutically acceptable salt or hydrate thereof to the intra-granular compressible filler is from about 3:1 to about 6:1.

5. The tablet of any one of claims 1-4, wherein the intra-granular compressible filler is present in an amount of from about 2 wt % to about 15 wt % by weight of the tablet.

6. The tablet of claim 5, wherein the intra-granular compressible filler is present in an amount of from about 3 wt % to about 10 wt % by weight of the tablet.

7. The tablet of any one of claims 1-6, wherein the compressible filler is microcrystalline cellulose.

8. The tablet of claim 7, wherein the microcrystalline cellulose is silicified microcrystalline cellulose.

9. The tablet of any one of claims 1-8, wherein the tablet is produced by a method comprising dry granulation process.

10. The tablet of any one of claims 1-9, further comprising from about 5 wt % to about 20 wt % of a low-compressibility filler.

11. The tablet of claim 10, wherein the low-compressibility filler is mannitol.

12. The tablet of any one of claims 1-11, further comprising from about 0.25 wt % to about 2 wt % of a binder. The tablet of claim 12, wherein the binder is polyvinylpyrrolidone.

14. The tablet of any one of claims 1-13, further comprising from about 25 wt % to about 80 wt % of an extra-granular compressible filler.

15. The tablet of claim 14, wherein the extra- granular compressible filler is anhydrous lactose.

16. The tablet of any one of claims 1-15, further comprising from about 2 wt % to about 3 wt % of a disintegrant.

17. The tablet of claim 16, wherein the disintegrant is croscarmellose sodium.

18. The tablet of any one of claims 1-17, further comprising from about 1 wt % to about 2 wt % of a lubricant.

19. The tablet of claim 18, wherein the lubricant is magnesium stearate.

20. A tablet comprising an intra-granular component and an extra-granular component, wherein the intra-granular component comprises Compound 1 and/or tautomers thereof or a pharmaceutically acceptable salt or hydrate thereof 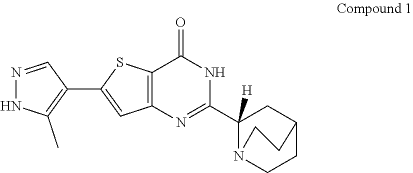 [SMILES: Cc1[nH]ncc1-c1cc2nc([C@@H]3CC4CCN3CC4)[nH]c(=O)c2s1]
and an intra-granular compressible filler.

21. The tablet of claim 20, wherein the weight ratio of Compound I and/or tautomers thereof or a pharmaceutically acceptable salt or hydrate thereof to the intra-granular compressible filler is greater than 1:1.

22. The tablet of claim 21, wherein the weight ratio of Compound I and/or tautomers thereof or a pharmaceutically acceptable salt or hydrate thereof to the intra-granular compressible filler is from about 2:1 to about 10:1.

23. The tablet of claim 22, wherein the weight ratio of Compound 1 and/or tautomers thereof or a pharmaceutically acceptable salt or hydrate thereof to the intra-granular compressible filler is from about 3:1 to about 6:1.

24. The tablet of any one of claims 20-23, wherein the intra-granular compressible filler is present in an amount from about 10 wt % to about 25 wt % by weight of the intra-granular component.

25. The tablet of claim 24, wherein the intra-granular compressible filler is present in an amount of from about 10 wt % to about 20 wt % by weight of the intra-granular component.

26. The tablet of any one of claims 20-25, wherein the intra-granular compressible filler is microcrystalline cellulose.

27. The tablet of claim 26, wherein the microcrystalline cellulose is silicified microcrystalline cellulose.

28. The tablet of any one of claims 20-27, wherein the tablet is produced by a method comprising dry granulation process.

29. The tablet of any one of claims 20-28, wherein the intra-granular component further comprises from about 20 wt % to about 40 wt % of a low-compressibility filler by weight of the intra-granular component.

30. The tablet of claim 29, wherein the low-compressibility filler is mannitol.

31. The tablet of any one of claims 20-30, wherein the intra-granular component further mprises from about 1 wt % to about 3 wt % of a binder by weight ©f the intra-granular component.

32. The tablet of claim 31 wherein the binder is polyvinylpyrrolidone.

33. The tablet of any one of claims 20-32, wherein the extra-granular component further comprises from about 90 wt % to 100 wt % of an extra-granular compressible filler by weight of the extra-granular component.

34. The tablet of claim 33, wherein the extra-granular compressible filler is anhydrous lactose.

35. The tablet of any one of claims 20-34, further comprising from about 2 wt % to about 3 wt % of a disintegrant by weight of the tablet, wherein the disintegrant is present in both the intra-granular component and the extra-granular component.

36. The tablet of claim 35, wherein e disintegrant is croscar nellose sodium.

37. The tablet of any one of claims 20-36, further comprising from about 1 wt % to about 2 wt % of a lubricant by weight of the tablet, wherein the lubricant is present in both the intra-granular component and the extra-granular component.

38. The tablet of claim 37, wherein the lubricant is magnesium stearate.

39. The tablet of any one of claims 20-38, wherein the weight ratio of the intra-granular component to the extra granular component is from about 1 :10 to about 3: 1.

40. The tablet of claim 39, wherein the weight ratio of the intra-granular component to the extra granular component is from about 1 :3 to about 2:1.

41. A tablet comprising:
from about 10 wt % to about 30 wt % of Compound 1 and/or tautomers thereof or a pharmaceutically acceptable salt or hydrate thereof 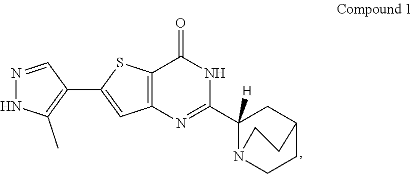 [SMILES: Cc1[nH]ncc1-c1cc2nc([C@@H]3CC4CCN3CC4)[nH]c(=O)c2s1]
from about 3 wt % to about 10 wt % of silicified microcrystalline cellulose,
from about 5 wt % to about 20 wt % of mannitol.
from about 0.25 wt % to about 2 wt % of polyvinylpyrrolidone,
from about 25 wt % to about 80 wt % of anhydrous lactose,
from about 2 wt % to about 3 wt % of crosearmellose sodium, and
from about 1 wt % to about 2 wt % of magnesium stearate.

42. The tablet of claim 41 wherein the tablet is produced by a method comprising dry granulation process.

43. A tablet comprising an intra-granular component and an e, iia-granularcomponent, whereine i a- granular component comprises:
from about 40 wt % to about 60 wt % of Compound 1 and/or tautomers thereof or a pharmaceutically acceptable salt or hydrate thereof 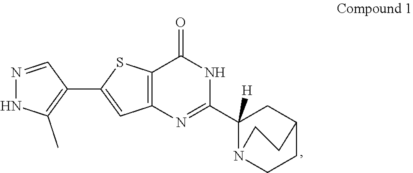 [SMILES: Cc1[nH]ncc1-c1cc2nc([C@@H]3CC4CCN3CC4)[nH]c(=O)c2s1]
from about 20 wt % of silicified microcrystalline cellulose,
from about 20 wt % to about 40 wt % of mannitol,
from about 1 wt % to about 3 wt % of polyvinylpyrrolidone,
from about 1 wt % to about 3 wt % of croscarmellose sodium. and
from about 0.5 wt % to about 2 wt % of magnesium stearate, and
wherein the extra-granular component comprises:
from about 90 wt % to about 98 wt % of anhydrous lactose,
from about 2 wt % to about 5 wt % of croscarmellose sodium, and
from about 1 wt % to about 3 wt % of magnesium stearate.

44. The tablet of claim 43, wherein the weight ratio of the intra-granular component to the extra granular component is from about 1:3 to about 2:1.

45. The tablet of claim 43 or 44, wherein the tablet is produced by a method comprising granulation process.

46. A tablet comprising:

| | |
|---|---|
| Compound 1 and/or tautomers thereof or a pharmaceutically | 12.5 wt % |
| acceptable salt or hydrate thereof | |
| 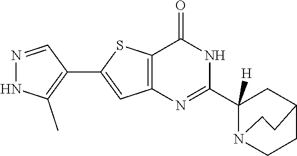 [SMILES: Cc1[nH]ncc1-c1cc2nc([C@@H]3CC4CCN3CC4)[nH]c(=O)c2s1] | |
| Compound 1 | |
| Mannitol | 7.5 wt % |
| Silicified microcrystalline cellulose | 3.75 wt % |
| Polyvinylpyrrolidone | 0.5 wt % |
| Croscarmellose sodium | 2.5 wt % |
| Magnesium stearate | 1.25 wt % |
| Anhydrous lactose | 72 wt % |

47. A tablet comprising:

| | |
|---|---|
| Compound 1 and/or tautomers thereof or a pharmaceutically | 25 wt % |
| acceptable salt or hydrate thereof | |
| 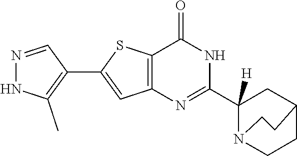 [SMILES: Cc1[nH]ncc1-c1cc2nc([C@@H]3CC4CCN3CC4)[nH]c(=O)c2s1] | |
| Compound 1 | |
| Mannitol | 15 wt % |
| Silicified microcrystalline cellulose | 7.5 wt % |
| Polyvinylpyrrolidone | 1 wt % |
| Croscarmellose sodium | 3 wt % |
| Magnesium stearate | 1.5 wt % |
| Anhydrous lactose | 47 wt % |
